(19) United States
(12) Patent Application Publication
(10) Pub. No.: US 2014/0000157 A1
(43) Pub. Date: Jan. 2, 2014

(54) CATALYTIC PROCESS FOR CONVERTING CARBON DIOXIDE TO A LIQUID FUEL OR PLATFORM CHEMICAL

(71) Applicant: ANTECY B.V., Hoevelaken (NL)

(72) Inventors: Paul O'CONNOR, HOEVELAKEN (NL); Sjoerd DAAMEN, BOCAIRENT (ES)

(73) Assignee: ANTECY B.V., Hoevelaken (NL)

(21) Appl. No.: 14/017,332

(22) Filed: Sep. 4, 2013

Related U.S. Application Data
(63) Continuation of application No. PCT/EP2012/053669, filed on Mar. 2, 2012.
(60) Provisional application No. 61/449,856, filed on Mar. 7, 2011.
(60) Provisional application No. 61/449,145, filed on Mar. 4, 2011.

(51) Int. Cl. C10L 1/02; C07C 45/00; C07C 51/00; C07C 29/151

(52) U.S. Cl. CPC C10L 1/02; C07C 29/1516; C07C 45/00; C07C 51/00
USPC 44/388; 568/840; 568/448; 562/609; 562/607

(57) ABSTRACT

A process for converting carbon dioxide to liquid fuels for a liquid fuel composition and/or a platform chemical composition. In this conversion process carbon dioxide is adsorbed to a catalyst composition, and reacted with hydrogen to form oxygenated hydrocarbons. Hydrogen for use in the process can be generated in situ or ex situ. The process can be carried out in a fully carbon neutral manner.

### CROSS-REFERENCE TO RELATED APPLICATIONS

This application is a continuation of PCT application number PCT/EP2012/053669 filed on 2 Mar. 2012, which claims priority from U.S. provisional application No. 61/449,145 filed 4 Mar. 2011 and U.S. provisional application No. 61/449,856 filed on 7 Mar. 2011. All applications are hereby incorporated by reference in their entireties.

### BACKGROUND OF THE INVENTION

1. Field of the Invention

The invention relates generally to a catalytic process in which carbon dioxide is reacted to a liquid fuel or platform chemicals, and more particularly to such a process wherein the catalyst composition is capable of adsorbing carbon dioxide.

2. Description of the Related Art

Crude-oil based liquid fuels are the backbone of the transportation infrastructure of the western world and, increasingly, of the developing world as well. These liquid fuels are associated with serious disadvantages. Many oil producing countries are politically unstable, with oil revenue being used to prop up undemocratic governments. The need to import large quantities of crude oil exposes mature economies to dramatic trade imbalances. And the combustion of crude oil based fuels is a major factor in the rising level of carbon dioxide in the earth's atmosphere, which is generally believed to contribute to global climate changes.

Thus, there is a growing interest in the development of alternative transportation fuels. Examples include plant-based oxygenated fuels, such as ethanol. Compressed natural gas, which is predominantly methane, can be used as a fuel for gasoline engines, requiring only modest modifications to the engine and the vehicle. Hydrogen is being used on a small scale, both as a fuel for internal combustion engines and for vehicles powered by one or mere fuel cells. More and more, electric power from the grid is being used as a transportation fuel, either in all-electric vehicles, such as the Nissan Leaf, or in so-called “plug-in hybrids”, such as the Chevrolet Volt.

Each of these alternative fuels presents serious disadvantages. Ethanol is generally produced from renewable sources, such as sugar and corn, which makes it in principle carbon neutral. Ethanol is also far less toxic than methanol. However, the fermentation processes used in producing fuel grade ethanol are expensive and time consuming. These processes produce ethanol/water mixtures containing large amounts of water, requiring energy-intensive separation steps. As a result the carbon gain from the use of ethanol is minimal, and the production costs are very high. At this time ethanol requires hefty subsidies for it to be able to compete with traditional gasoline. Ethanol is far more corrosive than gasoline. It can be blended with gasoline up to 15%; higher blending ratios would require significant modifications to vehicles and the distribution infrastructure.

Natural gas is domestically produced in a number of western countries, including Norway, The Netherlands, and the United States, and is abundantly available. Having a H/C ratio about twice that of gasoline, its combustion produces less carbon dioxide than gasoline. However, natural gas is a fossil fuel, and all carbon dioxide produced by its combustion represents a net increase of the amount of carbon dioxide in the atmosphere. In addition, the distribution and use of compressed natural gas as a transportation fuel would require a new infrastructure.

Hydrogen is currently produced from fossil fuels, which means that its use produces just the same amount of carbon dioxide as the direct combustion of fossil fuel. Hydrogen can be produced from renewable resources, such as solar energy and biomass. Therefore, hydrogen has the potential of offering carbon neutral energy. Its distribution and use would require an entirely new infrastructure, however.

The use of electric power from the grid has the advantage that the power is generated in centralized power plants, which offers the possibility of using renewable fuels, such as biomass, and/or carbon dioxide sequestration at the source. However, the use of electric power for propelling vehicles is inherently inefficient, because it requires the use of heavy batteries, which add to the energy required for moving the vehicle. In addition, storing and withdrawing electric energy in batteries results in significant losses.

Thus, there is a need for a process for converting energy from renewable resources to liquid fuels that are compatible with the existing liquid fuel infrastructure. There is a particular need for converting such energy to liquid hydrocarbons. There is an even greater need for such a process that uses carbon dioxide as one of the reactants.

### BRIEF SUMMARY OF THE INVENTION

The present invention addresses these problems by providing a process for converting carbon dioxide to a liquid fuel or platform chemicals, said process comprising the steps of:
a. providing a catalyst composition capable of adsorbing carbon dioxide;
b. contacting the catalyst composition with a carbon dioxide-containing gas stream, whereby carbon dioxide becomes adsorbed to the catalyst composition;
c. contacting the catalyst composition obtained in step b. with a hydrogen source;
d. supplying energy to the catalyst composition in the presence of the hydrogen source, whereby adsorbed carbon dioxide is catalytically reduced to a liquid fuel; Optionally the process comprises one or more of the following process steps:
e. Desorption of reaction products.
f. Regeneration of the catalyst.

The term “liquid fuel” as used herein includes, but is not limited to hydrocarbons. The term also includes, for example, methanol, which can be used “as is”, or can be converted to hydrocarbons. In the former case it is not fully compatible with the existing infrastructure for liquid transportation fuel distribution and consumption. The term also includes dimethyl ether (DME), which can be readily synthesized from methanol. DME, having a high cetane number, is an excellent alternative for conventional diesel fuel. Reaction products of the process can also be converted to longer chain hydrocarbons using a Fischer-Tropsch process. In this manner, conventional gasoline and diesel fuel fractions hydrocarbon mixtures can be produced.

The term “platform chemical” as used herein refers to any chemical compound that can be used as a feedstock in chemical synthesis reactions. As such, the term includes, but is not limited to, methanol, formaldehyde, formic acid, acetaldehyde, acetic acid, ethanol, and higher alcohols, such as butanol.

As a more general matter, the process of the invention has the potential of being entirely carbon neutral. For example, carbon dioxide used in the process may be obtained from the atmosphere, or from a flue gas. Hydrogen used in the process may be obtained from a renewable resource, such as biomass or solar energy. Energy used in any of the process steps may be from a renewable resource, such as solar energy. When operated in this manner, the process of the invention is fully carbon neutral, because no net carbon dioxide is produced in running the process, and the carbon dioxide produced when the liquid fuel is combusted does not exceed the amount of carbon dioxide consumed in the process.

As local or economic circumstances mandate, the process may also be run in what could be called a low carbon mode. For example, the hydrogen source used in step c. may be derived from a fossil fuel, or energy used in any of the process steps may be fully or partially derived from a non-renewable resource. Although in low carbon mode the process is not fully carbon neutral, it still offers a carbon efficiency that is much improved compared to processes based entirely on fossil fuels.

Another aspect of the invention comprises liquid fuel and platform chemicals produced by the inventive process.

### BRIEF DESCRIPTION OF THE DRAWINGS

The features and advantages of the invention will be appreciated upon reference to the following drawings, in which:

FIG. 1 is a schematic representation of a process embodiment in which hydrogen is generated in situ.

FIG. 2 is a schematic representation of a continuous process according to the embodiment of FIG. 1;

FIG. 3 is a schematic representation of the process of FIG. 2, modified in that an external hydrogen source is used.

FIG. 4 is a schematic representation of a process for simultaneously producing carbon dioxide and hydrogen for use in the process of the invention.

FIG. 5 is a schematic representation of an embodiment of the process in which water electrolysis is used as a hydrogen source.

FIG. 6 is a schematic representation of en embodiment of the process in which methane is used as a hydrogen source.

### DETAILED DESCRIPTION OF THE INVENTION

The catalyst composition used in the process of the invention is capable of adsorbing carbon dioxide. Materials capable of absorbing or adsorbing carbon dioxide are well known in the art. The processes of absorption and adsorption are fundamentally different. Absorption takes place throughout the bulk of the material, whereas adsorption is limited to the surface of the material. For the purpose of the process of the invention the interaction of the catalyst composition must be strong enough for the composition to sequester carbon dioxide from a gas stream (step b.), yet not so strong as to prevent the catalytic reaction with the hydrogen source (step d.) to take place.

As a general rule, materials that absorb carbon dioxide bind it too strongly for the purpose of step d. For this reason the catalyst composition preferably contains a material that adsorbs carbon dioxide. It will be understood that this rule is not absolute, as absorbent materials may be found that are capable of releasing the absorbed carbon dioxide in step d. These materials will be considered adsorbents of carbon dioxide within the meaning of step a. of the inventive process, even though the mechanism by which carbon dioxide is bound to the material may be one of absorption.

Suitable examples include alumina, layered double hydroxides, hydrotalcite, and hydrotalcite-like materials. The term “hydrotalcite-like materials” as used herein refers to materials having a crystal structure similar to that of hydrotalcite, wherein Mg ions are replaced with other divalent ions; or aluminum ions are replaced with other trivalent ions; or both. Examples of other suitable materials include zeolites, in particular zeolite Y and/or ZSM-5. The carbon dioxide adsorbent can suitable serve as catalyst support material as well.

It is desirable to include one or more catalytic metals in the catalytic composition, either in metallic form or as metal oxide. Examples of catalytic metals include Mn, Fe, Zn, Cu, Ce, Ni, Co, Cr, Pt, Ru, Sn, and combinations of these metals, for example Fe/Zn, Fe/Mn, Zn/Ce, and Cu/Zn. Particularly preferred catalytic compositions are those comprising ceria, for example ceria dispersed on a zeolite, such as zeolite Y and/or ZSM-5; and those comprising Cu/Zn, for example Cu/ZnO on an alumina or zeolite support. Other suitable support materials include (mixed) metal oxides comprising Mg, Ti, Zr, rare earth metals, such as Ce and mixed oxides of the perovskite type. It will be appreciated that some oxides, such as ceria, can act as (primarily) a catalytic support material or (primarily) as a catalyst, depending in particular on particle size. Catalytic support materials, such as mixed oxides containing ceria or zirconia will have oxygen storage capacity that can enhance the catalytic activity of the applied system. Small amount of stabilizers or dopants like Yttrium or Samarium can be added to improve the oxygen conductivity of the support and/or influence the oxygen capture/release dynamics of the supporting matrix.

Particularly preferred are nano-particulate and nano-porous forms of these catalytic metals and metal oxides.

The affinity of the adsorbent material for carbon dioxide can be increased by promoting the material with an alkaline earth or an alkali metal, in particular potassium. The catalytic activity of the metal component of the catalyst can be increased by the presence of the alkali metal.

In step b., the catalyst composition is contacted with a gas stream containing carbon dioxide, so that carbon dioxide becomes adsorbed to the catalyst composition. The gas stream may be ambient air, or it may be a gas that is enriched in carbon dioxide, for example gas streams comprising more than 5% w/w carbon dioxide. Examples of the latter include shale gas (a mixture of carbon dioxide and methane); gases obtained in the combustion of carbon-based fuel, such as engine exhaust; flue gas from a power plant, and the like. Carbon dioxide can be produced in other chemical reactions, such as a reform reaction of a hydrocarbon or coal, or in a water shift reaction. It is also possible to use a saturated carbon dioxide sequestering agent as the carbon dioxide source. The saturated sequestering agent is subjected to decomposition conditions, which causes it to release the sequestered carbon dioxide. The almost pure carbon dioxide stream is suitable for a highly efficient operation of step b.

The amount of gas that needs to be contacted with the catalyst composition in step b. is inversely related to the carbon dioxide content of the gas stream. If the gas stream is ambient air, a large volume of it needs to be passed over the catalyst composition in order to obtain the desired carbon dioxide loading. A significant amount of energy is required to flow this amount of air through a bed of the catalyst composition. However, it is possible to propel the air using solar energy, for example by means of a solar chimney.

The catalyst composition is contacted with a hydrogen source in step c. of the process. Step c. may be carried out subsequent to or concurrent with step b. The hydrogen source can be molecular hydrogen, or a hydrogen-containing compound, such as water or a hydrocarbon. Examples of suitable hydrocarbons include methane and ethane, methane being the preferred hydrocarbon. If water is used as the hydrogen source, it is typically used in the form of steam.

Molecular hydrogen can be produced by ex situ electrolysis of water, in a reform reaction of a hydrocarbon or coal, in a water gas shift reaction, and the like.

The choice of hydrogen source governs to some extent the choice of catalyst composition used in the process of the invention. As discussed above, the catalyst composition must be capable of adsorbing carbon dioxide. In addition, the catalyst composition must be capable of catalyzing the reaction of carbon dioxide with hydrogen, in step d. The latter function is suitably provided by metal sites in the catalyst composition, for example Cu, Zn, Cr, Ga, La and the lanthanides, Ni, and the like. Particularly preferred catalytic materials include CuO, ZnO, and mixtures thereof.

If a compound other than hydrogen is used as the hydrogen source, the catalyst must also be capable of dissociating the hydrogen source. If the hydrogen source is water, examples of suitable catalytic materials include Fe, Zn, Ni, Co, and the like. If the hydrogen source is a hydrocarbon, such as methane or ethane, suitable catalysts include the well-known Fischer Tropsch catalysts, such as Fe and Mn.

The catalytic conversion of step d. requires supplying energy to the catalyst composition in the presence of the hydrogen source. Energy can be supplied in the form of heat. Suitable reaction temperatures are in the range of from 100 to 1000° C., preferably from 200 to 750° C. The reaction rate can be increased by operating under elevated pressure, for example in the range of 5 to 200 bars, preferably from 10 to 50 bars.

In a preferred embodiment energy is supplied in the form of microwave energy. The use of microwave energy is particularly efficient, because it permits the reaction to be carried out at a lower overall temperature. Without wishing to be bound by theory, the inventors believe that the activation energy required for the reaction to proceed is supplied directly to the (metal) site of the catalyst, instead of by collisions with gas phase molecules, as would be the case in a thermal reaction. Accordingly, microwave energy provides “heat” where it is really needed, i.e., at the catalytic site where the reaction takes place, without a need to heat the entire reactor and its contents.

Thus, the reaction can be carried out at a lower temperature, but it will be appreciated that the concept of temperature does not have its conventional meaning in a reaction carried out under the influence of microwave energy. The more meaningful parameter is the amount of microwave energy supplied to the reaction mixture. This amount can be in the range of from 300 to 300,000 Watts/mole, preferably from 1000 to 200,000 Watts/mole.

In a preferred embodiment the energy supplied to the process is generated from a renewable resource. For example, solar energy can be used to generated electricity, either by photovoltaic means or by steam generated with solar heat. In a solar chimney the air flow can be used to drive a turbine, which in turn can generate electricity. In yet another embodiment, less valuable reaction products of the process of the invention can be burned to generate heat energy and/or electricity.

It will be understood that energy from a renewable resource can also be used to generate hydrogen, for example by using photovoltaic electricity to electrolyze water into oxygen and hydrogen.

After completion of step d. the catalyst can be regenerated by desorbing any reaction products adsorbed to it. The reaction products are carbon monoxide, methanol and methane. The reaction products may further contain higher alkanes, such as ethane, propane, and butane, in particular if the catalyst composition has Fischer-Tropsch activity. The reaction products can be desorbed from the catalyst by stripping the catalyst with an inert gas, such as steam.

Oxygen trapped on the catalyst particle, as may have been produced in in situ decomposition of water, can be removed from the catalyst using thermal or electromagnetic (e.g., microwave) energy. Ceria, or mixed oxides containing ceria can, in the reduced state, capture oxygen and at higher temperature releasing the captured oxygen forming a redox cycle, which can be represented by the following double equation.

Ce₂O₃+H₂O→2CeO₂+H₂, 2CeO₂+Energy→Ce₂O₃+0.5O₂

The temperature of the regeneration of the catalyst is generally higher than the temperature of the conversion step. Suitable reaction temperatures are in the range of 500-1500° C., preferably from 700-1200° C.

Alternatively reduction can be provoked at a lower temperature by applying a reducing agent like methane or higher hydrocarbons.

2CeO₂+CH₄→Ce₂O3+CO+2H₂,

The produced syngas (CO/H₂) can be fed back to step d of the process to be converted to a liquid hydrocarbon.

The skilled person will appreciate that the regenerated catalyst can be re-used in step b. Regeneration of the catalyst makes it possible to run the process continuously. It may be desired to cool down the catalyst prior to re-use in step b., in particular if step d. was carried out thermally (as distinguished from the use of microwave energy). Heat recovered from the catalyst in the cool down step can be re-used in one of the other process steps, in particular step d.

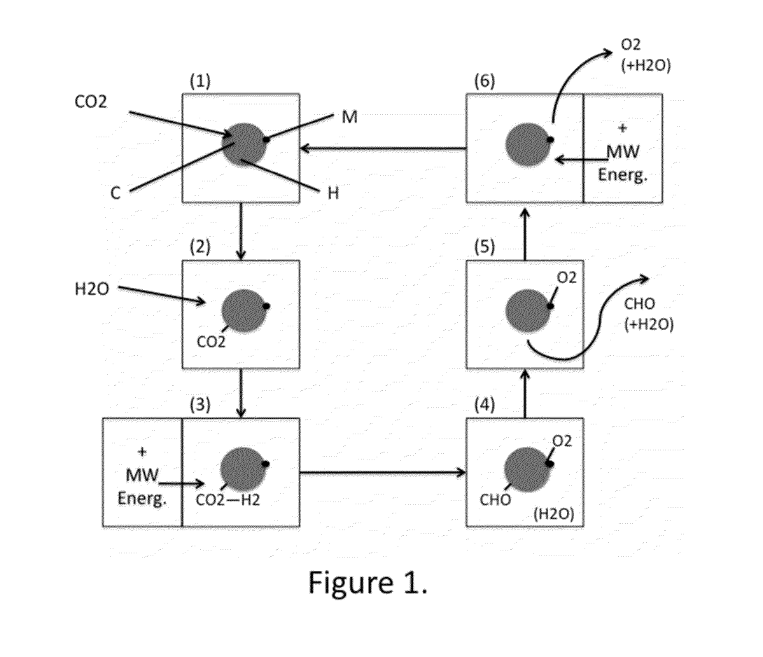

FIG. 1 shows a block diagram of an embodiment of the process of the invention. In block [1] catalyst composition C, comprising a carbon dioxide adsorbent material H and a metal component M, is contacted with a carbon dioxide containing gas stream, identified as CO₂. It will be understood that in reality the catalyst composition comprises a large number of metal particles M on each particle of carbon dioxide adsorbing material. It will also be understood that the adsorbent material is highly porous, so as to present a large specific surface area.

In block [2] the carbon dioxide laden catalyst composition is exposed to water in the form of steam. Under the influence of microwave energy MW, water decomposes on the catalytic surface to hydrogen (H₂) and oxygen (O₂) (see block [3]). Microwave energy continues to be applied in block [4]. In a preferred embodiment microwave energy is applied in a pulsed fashion. Adsorbed carbon dioxide reacts with hydrogen to form oxygenated hydrocarbons CHO, for example methanol (CH₃OH).

In block [5] reaction products CHO are stripped from the catalyst. In block [6] oxygen and any remaining water are removed from the catalyst by means of microwave energy. The catalyst is now ready to be recycled to block [1].

It will be appreciated that blocks [1] through [6] do not represent individual reactors. Rather, they represent individual stages of the process, which may all be carried out in one reactor, or in a number of consecutive reactors.

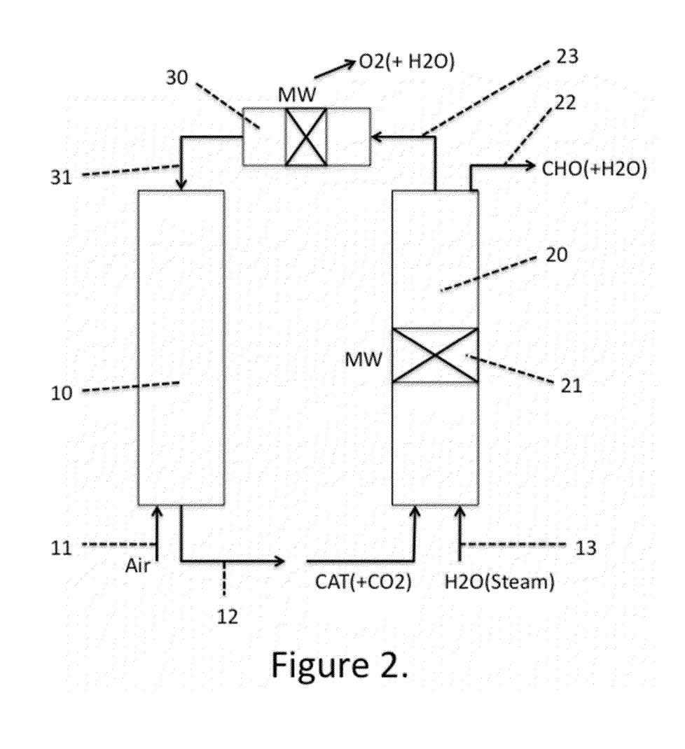

FIG. 2 provides a schematic representation of reactors in which the process of FIG. 1 can be carried out. A stream 31 of catalyst material enters first reactor 10 at the top, and flows down in countercurrent with air stream 11. In first reactor 10 the catalyst particles 31 adsorb carbon dioxide from air stream 11. The catalyst particles in first reactor 10 are fluidized or semi-fluidized by air stream 11, so that the residence time of the catalyst particles in first reactor 10 is optimized.

At the bottom of reactor 10 a stream 12 of carbon dioxide laden catalyst particles is collected and transported to second reactor 20. Second reactor 20 is a riser. Catalyst particles 12 are fluidized at the bottom by carrier gas 13, which comprises steam. The catalyst particles travel through zone 21 of second reactor 20. In zone 21 microwave energy is applied to the reaction mixture. In second reactor 20, in particular in zone 21, water is converted to hydrogen and oxygen, and carbon dioxide is converted to reaction products, such as methanol. Near the top of second reactor 20 the steam carrier gas acts to strip reaction products from the catalyst particles.

At the top of second reactor 20, a stream 22 of reaction products is separated from a stream 23 of catalyst particles. Stream 23 is conveyed to third reactor 30, where microwave energy is used to strip oxygen and water from the catalyst particles. The regenerated catalyst particles 31 are recycled to first reactor 10.

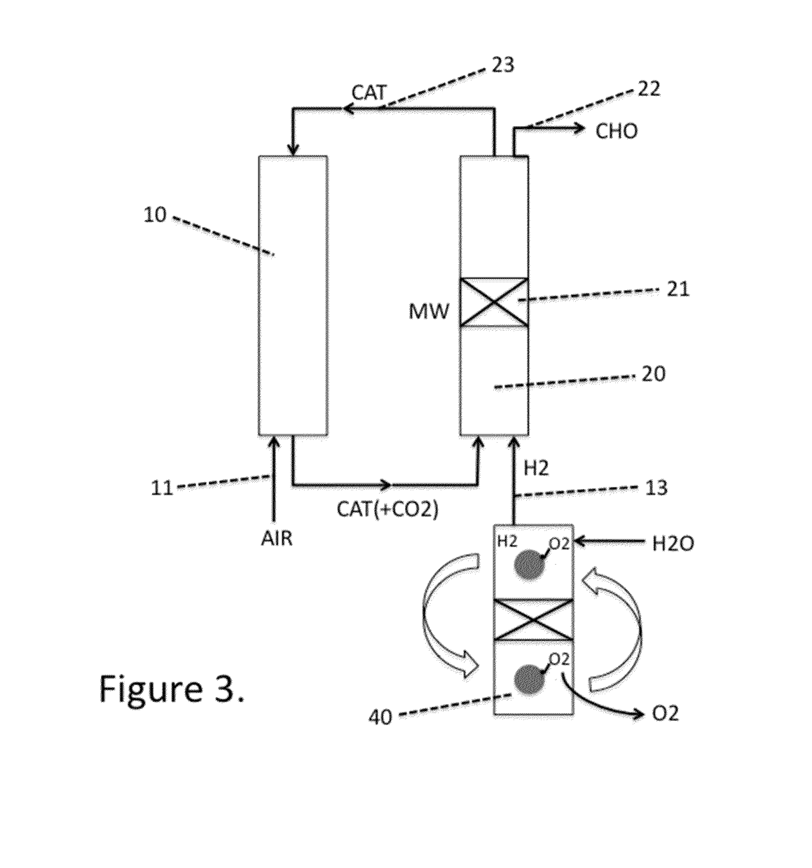

FIG. 3 shows an alternate embodiment of the reactors of FIG. 2. Instead of steam, carrier gas 13 introduced at the bottom of second reactor 20 comprises hydrogen. Hydrogen stream 13 is produced ex situ in hydrogen reactor 40. The plant of FIG. 3 does not contain a third reactor 30 for stripping oxygen from the catalyst. It will be understood that such a reactor can be included, if desired.

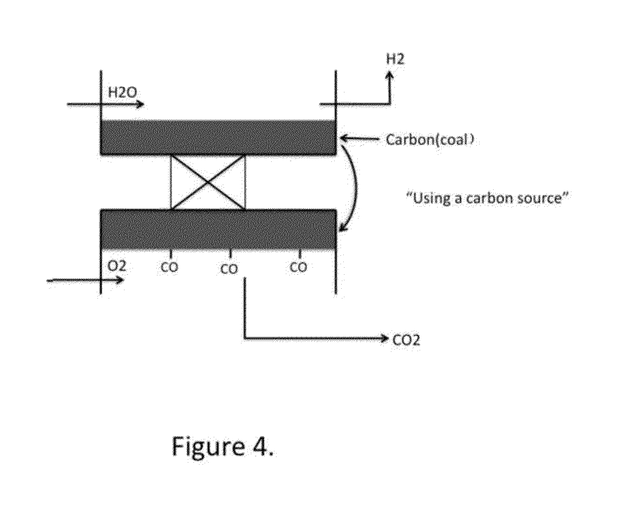

FIG. 4 is a schematic representation of a reform reactor, in which a carbon source, such as coal, is reacted to carbon dioxide and hydrogen.

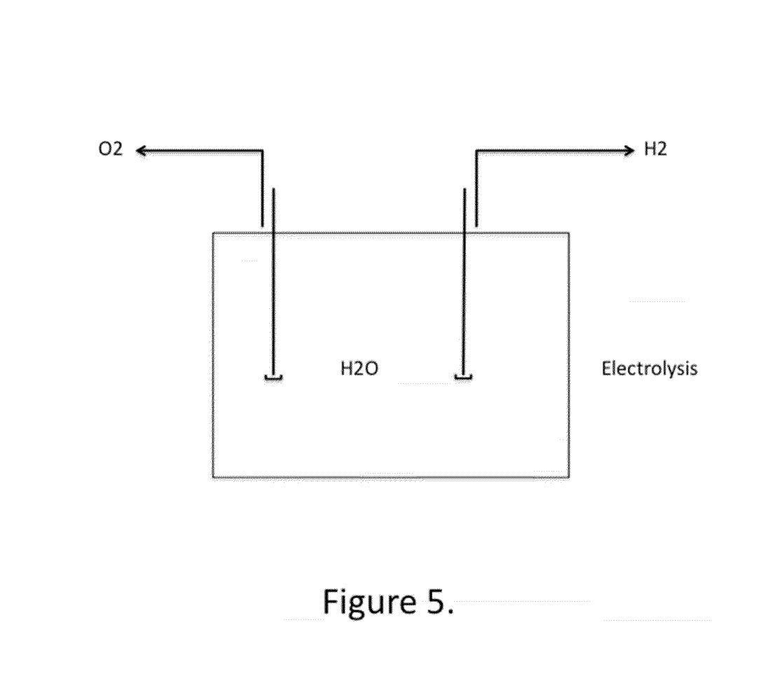

FIG. 5 is a schematic representation of an electrolysis cell, in which water is decomposed into oxygen and hydrogen, using electric energy.

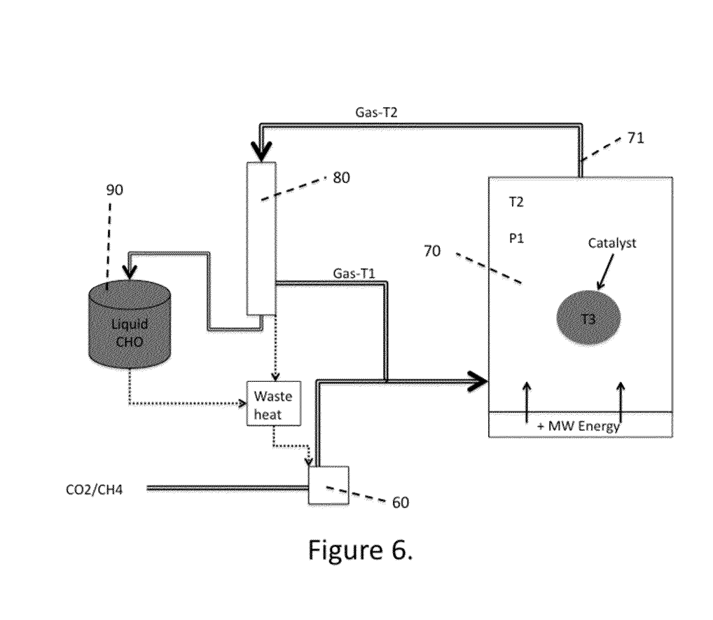

FIG. 6 shows an embodiment of the process of the invention specifically adapted for the conversion of shale gas, which is a mixture of primarily carbon dioxide and methane. Shale gas is preheated in heat exchanger 60. The preheated shale gas is pressurized to a pressure of 10-100 bar in reactor 70, which contains a bed of catalyst particles. The bulk temperature in reactor 70 is in the range of 200 to 300° C. Under influence of microwave radiation MW, the temperature of the catalyst particles is much higher, more than 400° C. The methane component of the shale gas mixture serves as a hydrogen source.

Product stream 71 consists primarily of oxygenated hydrocarbons, oxygen, water, unreacted methane, and unreacted carbon dioxide. This product stream is split in condensor 80 into liquid oxygenated hydrocarbons and gaseous products. The gaseous products are recycled to reactor 70. Waste heat recovered from condensor 80 is recycled to heat exchanger 60.

Thus, the invention has been described by reference to certain embodiments discussed above. It will be recognized that these embodiments are susceptible to various modifications and alternative forms well known to those of skill in the art.

Many modifications in addition to those described above may be made to the structures and techniques described herein without departing from the spirit and scope of the invention. Accordingly, although specific embodiments have been described, these are examples only and are not limiting upon the scope of the invention.

What is claimed is:

1. A process for converting carbon dioxide to a liquid fuel, said process comprising the steps of:
a) providing a catalyst composition capable of adsorbing carbon dioxide;
b) contacting the catalyst composition with a carbon dioxide-containing gas stream, whereby carbon dioxide becomes adsorbed to the catalyst composition;
c) contacting the catalyst composition obtained in step b) with a hydrogen source;
d) supplying energy to the catalyst composition in the presence of the hydrogen source, whereby adsorbed carbon dioxide is catalytically reduced to a liquid fuel;
e) regenerating the catalyst composition by desorption of reaction products.

2. The process of claim 1 which is made continuous by recycling the catalyst composition of step e) to step b).

3. The process of claim 1 wherein energy is supplied to the catalyst composition in step e).

4. The process of claim 1 wherein the energy used in step d) and/or step e) is generated from a renewable resource.

5. The process of claim 1 wherein the energy used in step d) and/or step e) comprises microwave energy.

6. The process of claim 5, wherein the microwave energy is pulsed microwave energy.

7. The process of claim 1 wherein the carbon dioxide-containing gas stream comprises atmospheric air.

8. The process of claim 7 wherein the carbon dioxide-containing gas stream is generated by a solar chimney.

9. The process of claim 8 wherein the energy supplied in step d) and/or step e) is generated by the solar chimney.

10. The process of claim 1 wherein the carbon dioxide containing gas stream comprises more than 5% w/w carbon dioxide.

11. The process of claim 10 wherein the carbon dioxide-containing gas stream is obtained in the combustion of a carbon-based fuel, in a reform reaction of a hydrocarbon or coal, or in a water gas shift reaction.

12. The process of claim 1 wherein the liquid fuel comprises methanol.

13. The process of claim 1 wherein the catalyst composition comprises Cu, Zn, Fe, Mn, Ni, Co, Cr, Pt, Ru, Sn, Ce, or a combination thereof.

14. The process of claim 13 wherein the catalyst composition comprises an oxide of Cu, Zn, Fe, Mn, Ce, or a combination thereof.

15. The process of claim 14 wherein the catalyst composition comprises CuO and/or ZnO.

16. The process of claim 14 wherein the catalyst composition further comprises an adsorbent for carbon dioxide selected from alumina, layered double hydroxides, hydrotalcite and hydrotalcite-like materials.

17. The process of claim 16 wherein the adsorbent for carbon dioxide is promoted with an alkali metal.

18. The process of claim 17 wherein the alkali metal is potassium.

19. The process of claim 1 wherein the hydrogen source comprises molecular hydrogen.

20. The process of claim 19 wherein the molecular hydrogen is obtained by electrolysis of water.

21. The process of claim 20 wherein the water electrolysis is carried out with energy from a renewable resource.

22. The process of claim 19 wherein the molecular hydrogen is obtained in a reform reaction of a hydrocarbon or coal, or in a water gas shift reaction.

23. The process of claim 1 wherein the hydrogen source is water, and hydrogen is created in the presence of the catalyst composition by supplying energy.

24. The process of claim 23 wherein the energy is supplied at least in part in the form of microwave energy.

25. A liquid fuel composition produced by the process of claim 1.

26. A platform chemical composition produced by the process of claim 1.
